(19) United States
(12) Patent Application Publication
(10) Pub. No.: US 2011/0022062 A1
(43) Pub. Date: Jan. 27, 2011

(54) Application instrument for an aneurysm clip

(75) Inventors: Olaf Hegemann, Tuttlingen (DE); Dieter Weisshaupt, Immendingen (DE); Markus Nesper, Tuttlingen (DE); Laligam N. Sekhar, Seattle, WA (US)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/655,557

(22) Filed: Dec. 30, 2009

(30) Foreign Application Priority Data
Jul. 22, 2009 (DE) 10 2009 034 142

(51) Int. Cl. A61B 17/10

(52) U.S. Cl. 606/142

(57) ABSTRACT

In order, in an application instrument for an aneurysm clip comprising two clamping jaws, which are movable relative to one another and between them accommodate the aneurysm clip, as well as actuating elements for moving the clamping jaws towards and away from one another and thereby for opening and closing the aneurysm clip, to assist the surgeon with the controlled closing movement of the aneurysm clip, it is proposed that the application instrument comprises a damping element, which during closing of the aneurysm clip imposes a retarded mutual movement of the clamping jaws.

The invention relates to an application instrument for an aneurysm clip comprising two clamping jaws, which are movable relative to one another and between them accommodate the aneurysm clip, as well as actuating elements for moving the clamping jaws towards and away from one another and thereby for opening and closing the aneurysm clip.

Application instruments for aneurysm clips are used to open aneurysm clips, i.e. move the clamping jaws of the aneurysm clip away from one another, and to apply these aneurysm clips to a vessel of a patient in such a way that the clamping jaws of the aneurysm clip close the part of the vessel situated between the clamping jaws. Here, it is essential that the closing movement of the aneurysm clip is effected slowly and uniformly as a sudden closing of the aneurysm clip may lead to damage of the vessel and in the worst-case scenario even to tearing of the vascular wall. Normally, this uniform and slow closing of the aneurysm clip is controlled by the actuating elements, which the surgeon actuates, for example in the case of actuating elements in the form of handles of a forceps-shaped application instrument. This requires a great deal of delicacy on the part of the surgeon, and there is a risk that inadvertently too rapid a closing movement may nevertheless occur.

The object of the invention is to design an application instrument of the above type in a way that assists the surgeon with the slow and uniform closing of the clamping jaws of the aneurysm clip.

In an application instrument of the type described in the introduction this object is achieved according to the invention in that the application instrument comprises a damping element, which during closing of the aneurysm clip imposes a retarded mutual movement of the clamping jaws of the application instrument.

In this way it is ensured that even in the event of a rapid movement of the actuating elements of the application instrument this rapid movement is not transmitted directly to the clamping jaws of the application instrument, rather by means of the damping element these clamping jaws are retarded and also moved uniformly relative to one another. The end result is a correspondingly uniform and retarded closing movement of the aneurysm clip held between the clamping jaws of the application instrument.

It is particularly advantageous if during opening of the aneurysm clip the damping element is ineffective so that during opening of the aneurysm clip the clamping jaws move without being retarded. Thus, for opening of the aneurysm clip the surgeon may use the application instrument in the same way as he is accustomed to using one without a damping element, but during closing of the aneurysm clip the damping element comes into effect and decelerates this closing movement.

In a preferred embodiment it is provided that the damping element comprises a hydraulically or pneumatically operating damping element.

For example, the damping element may comprise a flow path for a hydraulic or pneumatic medium, in which a throttle is disposed, wherein during closing of the aneurysm clip the damping element feeds hydraulic or pneumatic medium through said flow path. The throttle in the flow path then ensures that the flow rate is reduced and rendered uniform, with the result that the closing of the aneurysm clip also occurs in a retarded and uniform manner.

In this case, it is particularly advantageous if there is associated with the flow path a bypass line, which bypasses the throttle and in which is disposed a one-way valve, which during closing of the aneurysm clip is closed and during opening of the aneurysm clip is open. Thus, the throttle is effective only during closing, not however during opening, of the aneurysm clip, i.e. a deceleration of the movement of the clamping jaws of the application instrument occurs during closing, not however during opening, of the aneurysm clip.

In a first preferred embodiment it may be provided that the actuating elements of the clamping jaws upon actuation feed the hydraulic or pneumatic medium through the flow path. The movement of the actuating elements therefore leads to the hydraulic or pneumatic medium flowing through the flow path.

In another preferred embodiment it may be provided that the actuating elements are actuable by supplying the hydraulic or pneumatic medium, which medium in this case flows through the flow path. Here, it is therefore a case of a driving of the actuating elements by means of the hydraulic or pneumatic medium, wherein the flowing of the hydraulic or pneumatic medium through the flow path ensures that this movement of the actuating elements and hence the movement of the clamping jaws of the application instrument during closing of the aneurysm clip are effected in a retarded manner.

In particular, the damping element may comprise a piston-cylinder unit.

In a preferred embodiment it is provided that the actuating elements take the form of handles of a forceps-like application instrument and the damping element is disposed between the handles. Thus, the damping element does not impede either the manipulation or the view of the application region of the application instrument and may nevertheless ensure a retarded movement of the clamping jaws during closing of the aneurysm clip.

The following description of preferred embodiments of the invention serves in conjunction with the drawings to provide a more detailed explanation. The drawings show:

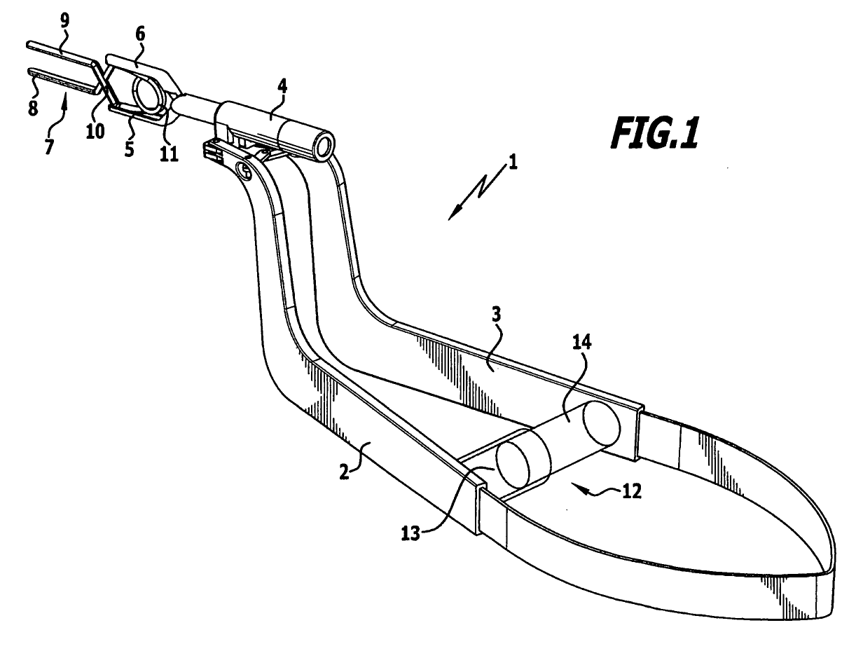

FIG. 1: a perspective view of an application instrument for an aneurysm clip having a damping element between the handles;

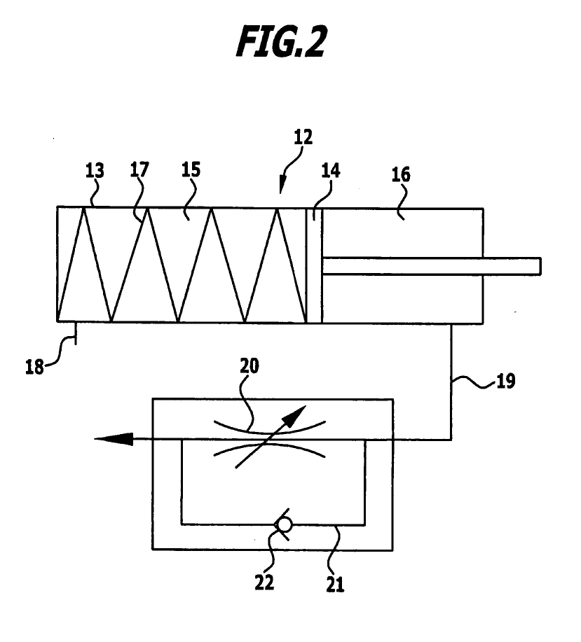

FIG. 2: a diagrammatic representation of a pneumatic damping element;

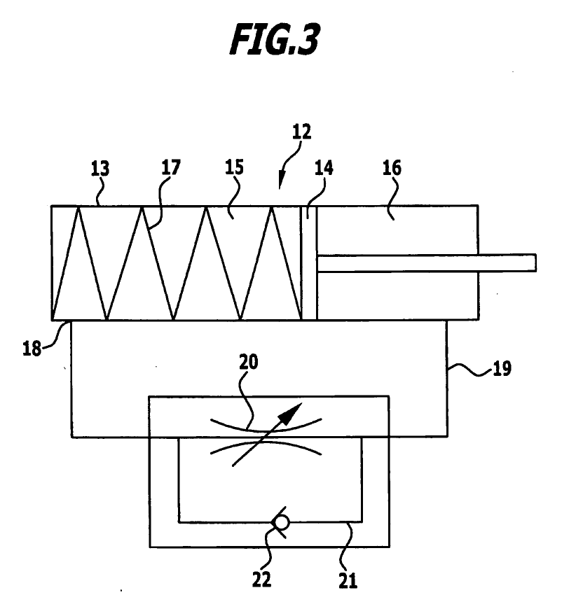

FIG. 3: a view similar to FIG. 2 of a hydraulic or pneumatic damping element;

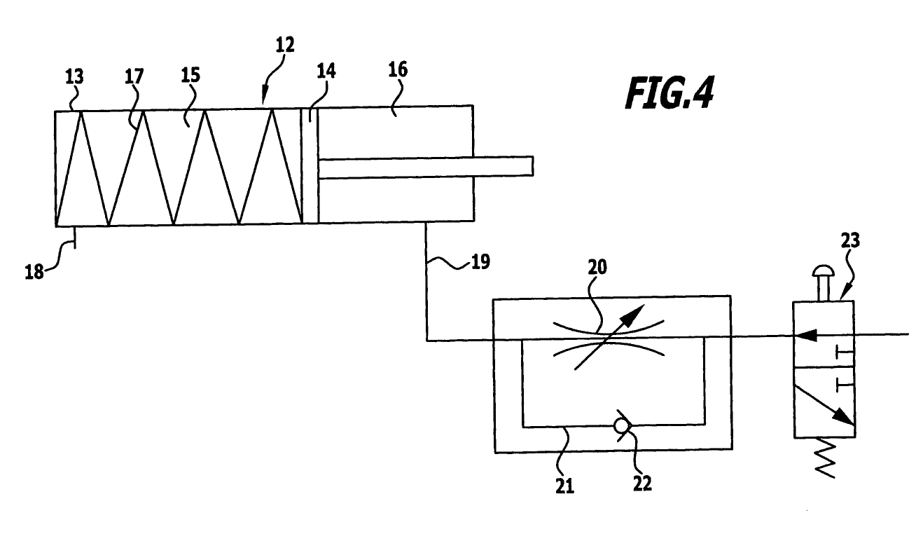

FIG. 4: a view of a pneumatic damping element that simultaneously generates the movement of the clamping jaws of the application instrument, and

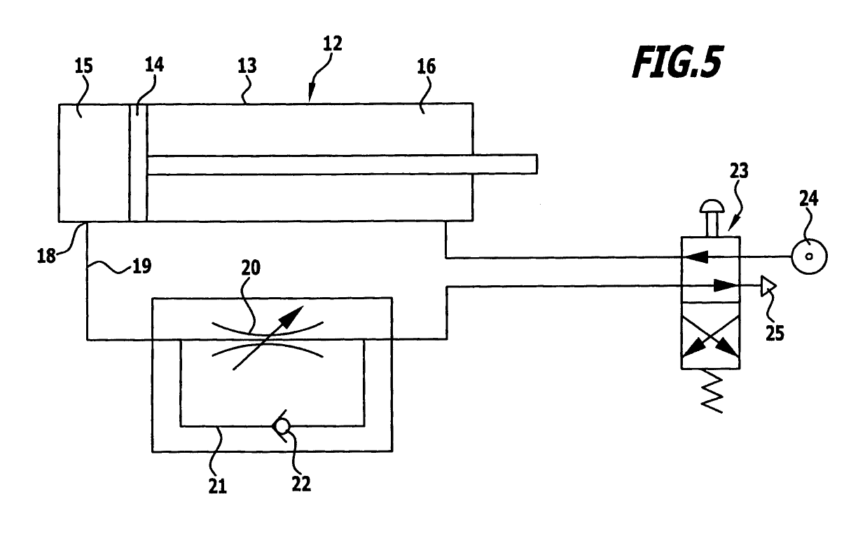

FIG. 5: a view similar to FIG. 4 of a hydraulic or pneumatic damping element.

The application instrument 1 illustrated in FIG. 1 comprises two handles 2, 3, which are pivotable relative to one another and are mounted pivotably on a housing 4. The housing 4 carries on its distal end two clamping jaws 5, 6, which extend substantially parallel to one another and are movable by means of a mechanism not shown in the drawing in such a way relative to one another that their spacing varies. The movement of the clamping jaws 5, 6 is produced in this case by pivoting the handles 2, 3 relative to one another, this movement being transmitted from the handles 2, 3 to the clamping jaws 5, 6 by means of a gear mechanism in the interior of the housing 4 that is not shown in the drawing. In this way it is possible for example, by pressing the handles 2, 3 together, to move the clamping jaws 5, 6 away from one another and conversely, by pivoting the handles 2, 3 apart from one another, to move the clamping jaws 5, 6 towards one another.

The design of the application instrument of FIG. 1 is only one example of such an application instrument, the significant point being merely that such an application instrument 1 comprises two clamping jaws 5, 6 that are movable relative to one another, which are movable in some way relative to one another. For example, it would also be possible for the clamping jaws to be held directly on extensions of the handles that are pivotable relative to one another, so that by means of a pivoting movement of the handles the clamping jaws are pivotable relative to one another and hence their mutual spacing is variable.

Inserted between the clamping jaws 5, 6 is an aneurysm clip 7. This comprises two clamping arms 8, 9, which extend parallel to one another and which after a crossover 10 are connected to one another by a coiled curved connecting portion 11. This coiled connecting portion 11 acts as a closing spring, which normally presses the two clamping arms 8, 9 resiliently towards one another. By means of pressure upon the portions of the clamping arms 8, 9 immediately adjoining the connecting portion 11, the clamping arms 8, 9 in the distal region of the aneurysm clip 7 may be moved away from one another, i.e. the aneurysm clip is opened by means of a pressure upon those portions of the clamping arms 8, 9 that immediately adjoin the connecting portion 11, whereas without such a pressure the aneurysm clip is closed only under the action of the connecting portion 11.

The aneurysm clip 7 inserted between the clamping jaws 5, 6 of the application instrument 1 lies with the portions of its clamping arms 8, 9 that are adjacent to the connecting portions 11 against the clamping jaws 5, 6, so that the clamping jaws 5, 6 exert a pressure on these portions when the clamping jaws 5, 6 are moved towards one another. This leads to an opening of the aneurysm clip, while an increase of the spacing of the clamping jaws 5, 6 leads to a closing of the aneurysm clip.

Disposed between the handles 2, 3 is a piston-cylinder unit 12, wherein the cylinder 13 of the piston-cylinder unit 12 is held on one handle 2, while the piston 14 of the piston-cylinder unit 12 that engages into the cylinder 13 is held on the other handle 3. The piston 14 is guided in a sealed manner in the cylinder 13 and subdivides the interior of the cylinder 13 into two chambers 15, 16, which are sealed relative to one another. Disposed in one of the two chambers 15 is a spring, for example a helical spring, which displaces the piston 14 normally into the extended position, i.e. into a position, in which the chamber 16 has a low volume and the chamber 15 has a high volume. The piston 14 may be pushed into the cylinder 13 counter to the action of the spring 17.

In a first embodiment, which is shown in FIG. 2, the chamber 15 is connected to the environment by a vent hole 18, while the other chamber 16 is connected by a flow line 19, in which a throttle 20 is inserted. A bypass line 21 bypasses the throttle 20 and there is situated in this bypass line 21 a one-way valve 22, which is closed when air flows out of the chamber 16 but open when air flows in.

This arrangement forms a damping element, which upon a movement of the piston 14 counter to the action of the spring 17 allows an unimpeded flow of air into the chamber 16, but given the reverse movement the flow of air out of the chamber 16 is impeded by the throttle 20 because in the case of the outward flow the one-way valve 22 is closed and all of the air has to flow through the throttle 20. As a result of this, this movement of the piston 14 is effected in a retarded and damped manner, i.e. the movement is slowed down and moreover additionally rendered uniform. Even if the surgeon, after pivoting the handles 2, 3 towards one another and hence opening the aneurysm clip 7, simply lets go of the handles 2, 3, the handles 2, 3 under the action of the spring 17 are pivoted only slowly apart from one another, and the result is that the clamping jaws 5, 6 of the application instrument 1 are also moved only slowly away from one another. The closing movement of the aneurysm clip 7 thus generated is therefore effected in a retarded and uniform manner.

In the embodiment of FIG. 3, instead of a pneumatic medium a hydraulic medium is introduced into the piston-cylinder unit, the flow line 19 in this case being connected directly to the vent hole 18, so that the hydraulic medium upon displacement of the piston 14 is fed from the one chamber 15 into the other chamber 16 and vice versa. The effect of the throttle 20 and the bypass line is however the same so that, in this case too, a retarded and uniform closing movement of the aneurysm clip is generated.

In the previously described embodiments the closing movement of the clamping jaws 5, 6 is generated by the surgeon as a result of manual actuation of the handles 2, 3. In the embodiment of FIGS. 4 and 5, however, this movement of the clamping jaws 5, 6 is effected by means of an active damping element, which in turn is actuated pneumatically or hydraulically.

In the case of the embodiment of FIG. 4, which is provided for pneumatic operation, a similar construction of the piston-cylinder unit 12, the flow line 19 with throttle 20 and the one-way valve 22 is selected but the flow line 19 does not open out in the environment but is connected by a changeover valve 23 to a compressed-gas source, for example a compressed-air source. When compressed air is supplied from this compressed-air source, this compressed air in a first position of the changeover valve 23 flows through the flow line 19 and the throttle 20 into the chamber 16 and therefore displaces the piston 14 counter to the action of the spring 17. This inward flow is effected substantially through the bypass line 21 because during the inward flow the one-way valve 22 is opened and offers a lower resistance to the gas flow than the throttle 20.

In the second position of the changeover valve 23 the flow line 19 is no longer connected to the compressed-gas source but opens out into the environment. As a result, the gaseous medium enclosed in the chamber 16 may flow out through the flow line 19, now however, in the closed state of the one-way valve 22, exclusively through the throttle 20 and hence in a retarded and uniform manner. This movement is used for the closing movement of the aneurysm clip, so that for opening of the aneurysm clip by substantially bypassing the throttle a rapid movement of the clamping jaws 5, 6 is possible, while for closing of the aneurysm clip 7 a retarded and uniform movement is possible.

In the embodiment of FIG. 5, a hydraulic medium that is supplied from a pressure source 24 is used to drive the piston 14.

The arrangement is selected in a similar manner to that in the embodiment of FIG. 3, except that an interruption is inserted into the flow line 19 between the chamber 16 and the branch of the bypass line 21 in that the part of the flow line 19 leading to the chamber 16 is connected to the pressure source 24, while the part of the flow line 19 leading to the branch of the bypass line 21 is connected to a collecting tank 25 for the hydraulic medium. These connections are established by means of a changeover valve 23, which in a first position connects the chamber-side part of the flow line 19 to the pressure source 24 and the throttle-side part of the flow line 19 to collecting tank 25 but in a second position establishes the connections the other way round, so that then the chamber-side part of the flow line 19 is connected to the collecting container 25 and the throttle-side part of the flow line 19 is connected to the pressure source 24.

In the first position represented in FIG. 5, the pressure source feeds hydraulic medium into the chamber 16 and therefore displaces the piston 14 counter to the action of the spring 17, in which case hydraulic medium leaves the chamber 15 and passes through the open one-way valve 22 and to a slight extent also through the throttle 20 into the collecting tank 25. Thus, in this position of the changeover valve the flow in the flow line 19 is not impeded by the throttle 20, this corresponding to the opening movement of the aneurysm clip.

In the second position of the changeover valve 23, the hydraulic medium from the pressure source 24 passes, in the closed state of the one-way valve 22, exclusively through the throttle 20 into the chamber 15 and in this case displaces the piston 14 in the direction of the chamber 16, while the hydraulic medium stored in the chamber 16 is fed through the chamber-side part of the flow line 19 to the collecting tank 25. This flow movement is slowed down and rendered uniform by the throttle 20, this corresponding to the closing movement of the aneurysm clip.

This closing movement is therefore determined by means of the throttle 20, and of course it would additionally be possible, by controlling the pressure of the fluid supplied from the pressure source 24, additionally to influence the closing movement of the aneurysm clip. This might be effected for example by additionally inserting a metering valve into the flow line or by correspondingly varying the feed of fluid from the pressure source.

Thus, in the embodiments of FIGS. 2 and 3 a passive damping element is used, which assists the manual actuation of the handles 2 and 3 by the surgeon and during closing of the aneurysm clip prevents too rapid a movement of the clamping jaws 5, 6, while in the embodiment of FIGS. 4 and 5 an active damping element is used, which simultaneously forms the drive for the movement of the clamping jaws 5, 6.

1-10. (canceled)

11. Application instrument for an aneurysm clip comprising two clamping jaws, which are movable relative to one another and between them accommodate the aneurysm clip, as well as actuating elements for moving the clamping jaws towards and away from one another and thereby for opening and closing the aneurysm clip, wherein the application instrument comprises a damping element, which during closing of the aneurysm clip imposes a retarded mutual movement of the clamping jaws.

12. Application instrument according to claim 11, wherein during opening of the aneurysm clip the damping element is ineffective so that during opening of the aneurysm clip the clamping jaws move without being retarded.

13. Application instrument according to claim 11, wherein the damping element comprises a mechanically effective damping element.

14. Application instrument according to claim 12, wherein the damping element comprises a mechanically effective damping element.

15. Application instrument according to claim 11, wherein the damping element comprises a hydraulically or pneumatically operating damping element.

16. Application instrument according to claim 12, wherein the damping element comprises a hydraulically or pneumatically operating damping element.

17. Application instrument according to claim 15, wherein the damping element comprises a flow path for a hydraulic or pneumatic medium, in which a throttle is disposed, and wherein during closing of the aneurysm clip the damping element feeds hydraulic or pneumatic medium through said flow path.

18. Application instrument according to claim 16, wherein the damping element comprises a flow path for a hydraulic or pneumatic medium, in which a throttle is disposed, and wherein during closing of the aneurysm clip the damping element feeds hydraulic or pneumatic medium through said flow path.

19. Application instrument according to claim 17, wherein there is associated with the flow path a bypass line, which bypasses the throttle and in which is disposed a one-way valve, which during closing of the aneurysm clip is closed and during opening of the aneurysm clip is open.

20. Application instrument according to claim 18, wherein there is associated with the flow path a bypass line, which bypasses the throttle and in which is disposed a one-way valve, which during closing of the aneurysm clip is closed and during opening of the aneurysm clip is open.

21. Application instrument according to claim 17, wherein the actuating elements of the clamping jaws upon actuation feed the hydraulic or pneumatic medium through the flow path.

22. Application instrument according to claim 18, wherein the actuating elements of the clamping jaws upon actuation feed the hydraulic or pneumatic medium through the flow path.

23. Application instrument according to claim 17, wherein the actuating elements are actuable by supplying the hydraulic or pneumatic medium, which medium in this case flows through the flow path.

24. Application instrument according to claim 18, wherein the actuating elements are actuable by supplying the hydraulic or pneumatic medium, which medium in this case flows through the flow path.

25. Application instrument according to claim 17, wherein the damping element comprises a piston-cylinder unit.

26. Application instrument according to claim 11, wherein the actuating elements take the form of arms of a forceps-like application element and the damping element is disposed between the arms.
